(19) United States
(12) Patent Application Publication
(10) Pub. No.: US 2013/0295062 A1
(43) Pub. Date: Nov. 7, 2013

(54) METHODS AND COMPOSITIONS FOR TREATMENT OF ALZHEIMER'S DISEASE

(71) Applicants: Kenneth E. Bernstein, Los Angeles, CA (US); Maya Koronyo, Los Angeles, CA (US); Sebastien Fuchs, Los Angeles, CA (US); Yosef Koronyo, Los Angeles, CA (US)

(72) Inventors: Kenneth E. Bernstein, Los Angeles, CA (US); Maya Koronyo, Los Angeles, CA (US); Sebastien Fuchs, Los Angeles, CA (US); Yosef Koronyo, Los Angeles, CA (US)

(73) Assignee: CEDARS-SINAI MEDICAL CENTER, Los Angeles, CA (US)

(21) Appl. No.: 13/979,610

(22) PCT Filed: Jan. 18, 2012
(86) PCT No.: PCT/US2012/021764

§ 371 Date: Jul. 12, 2013

Related U.S. Application Data
(60) Provisional application No. 61/433,895, filed on Jan. 18, 2011.

(51) Int. Cl. A61K 38/48

(52) U.S. Cl. CPC A61K 38/4813
USPC 424/93.7; 424/94.63; 424/93.21

(57) ABSTRACT

The present invention relates to methods and compositions for use in the treatment of neurological disorders, such as Alzheimer's disease and dementia. In one embodiment, the present invention provides a method of treatment of Alzheimer's disease in an individual by administering a composition overexpressing ACE to the individual. In another embodiment, the method of treatment comprises administering a composition comprising a therapeutically effective dosage of ACE.

### CROSS-REFERENCE TO RELATED APPLICATIONS

This application claims priority to U.S. Provisional Application No. 61/433,895 filed Jan. 18, 2011, which is hereby incorporated by reference in its entirety.

### FIELD OF THE INVENTION

The present invention relates generally to Alzheimer's disease and neurological disorders, and more specifically to methods and compositions for use in the treatment of Alzheimer's disease and neurological disorders.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Alzheimer's disease (AD) is a devastating, age-related neurodegenerative disorder, the most frequent form of senile dementia and the sixth-leading cause of death in the U.S. More than 5 million Americans are already facing the challenges of AD and more than twice that number are caring for them. In the near future, an additional 10 million American baby boomers are predicted to join the ranks of the afflicted. It is a progressive, incurable and ultimately fatal neurodegenerative disorder.

While the exact etiology of this disease is not fully understood, one of the prominent hypotheses is that brain pathology is due to the accumulation of amyloid-β(Aβ) peptides, in their soluble and aggregated (Aβ0 plaques) forms. However, currently AD is terminal and despite considerable progress in understanding the biology of AD, an effective treatment still remains elusive. These facts present an urgent need to find new approaches for AD and dementia, and a significant need in the art for the development of novel and effective treatments.

### SUMMARY OF THE INVENTION

Various embodiments include a method of treating a neurological disease and/or condition in a subject, comprising providing a therapeutically effective dosage of a composition modified to express angiotensin convening enzyme (ACE), or a pharmaceutical equivalent, derivative, analog or salt thereof, and administering the therapeutically effective dosage of the composition to the subject. In another embodiment, the composition comprises monocytes, macrophages and/or neuroglial cells. In another embodiment, the composition is administered to the subject intravenously. In another embodiment, the composition is administered to the subject by direct injection. In another embodiment, the subject is a human. In another embodiment, the subject is a rodent. In another embodiment, the composition has been derived from the bone marrow of the subject. In another embodiment, the composition has been modified in vitro to express ACE, or a pharmaceutical equivalent, derivative, analog or salt thereof. In another embodiment, ACE or a pharmaceutical equivalent, derivative, analog or salt thereof, degrades amyloid beta peptides in the subject. In another embodiment, the neurological disease and/or condition is Alzheimer's disease. In another embodiment, expression of ACE or a pharmaceutical equivalent, derivative, analog or salt thereof takes place in the brain of the subject.

Other embodiments described herein include a composition comprising one or more cells genetically modified to selectively overexpress angiotensin converting enzyme (ACE) or a pharmaceutical equivalent, derivative, analog or salt thereof. In another embodiment, the one or more cells are myelomonocytic cells.

Other embodiments herein include a composition, comprising a therapeutically effective amount of angiotensin converting enzyme (ACE), or a pharmaceutical equivalent, derivative, analog or salt thereof, and a pharmaceutically acceptable carrier.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment herein, plaques in ACE wild type non-Alzheimer's Mouse (a) cortex and (e) hippocampus, APP/PS1 (B6.Cg-Tg(APPswe, PSEN1ΔE9) 85 Dbo/J) mice having WT ACE alleles (b) cortex and (f) hippocampus. APP/PS1 mice with one ACE 10 allele (ACE 10/WT) (c) cortex and (g) hippocampus, and APP mice with two ACE 10 alleles (ACE 10/10) (d) cortex and (h) hippocampus. There is a noticeable reduction of plaques with either one or two alleles of ACE 10.

FIG. 2 depicts, in accordance with an embodiment herein, a quantitation showing the plaque area in the (a) hippocampus and (b) cortex of the 4 groups of mice described in FIG. 1 above. Mice with one or two ACE 10 alleles have reduced brain plaque. Thus, increasing expression of ACE in macrophages and microglia reduces the plaque area.

FIG. 3 depicts, in accordance with an embodiment herein, decreased plaque numbers and size in mice having one or two alleles of the ACE 10 gene, using the antibody 6E10. (a) describes hippocampus, plaque area, (b) describes hippocampus, plaque number, (c) describes cortex, plaque area, and (d) describes cortex, plaque number, in total demonstrating that increasing expression of ACE in macrophages and microglia reduces the plaque area and the plaque number in both hippocampus and cortex.

FIG. 4 depicts, in accordance with an embodiment herein, assays in (a) brain, and (b) plasma. These are assays characterizing the soluble levels of the Aβ1-42 protein in the brain and in the blood, showing that mice having one or two ACE 10 alleles have reduced quantities of the pathogenic protein in both the brain and the blood. The reduction in levels of this protein correlates with the reduction of plaque size and number.

FIG. 5 depicts, in accordance with an embodiment herein, cerebral AP plaques stained with Thio-S and 6E10 mAb, in AD-Tg mice with wt ACE expression (AD+ACEwt/wt) as compared to AD-Tg mice with ACE overexpression in microglia and monocyte/MΦ (AD+ACE10/10).

FIG. 6 depicts, in accordance with an embodiment herein, quantitative analysis of Aβ plaque area in the hippocampus and cortex, stained with 6E10. Mice with one or two ACE 10 alleles have significant reduction of plaques area.

FIG. 7 depicts, in accordance with an embodiment herein, thio-S quantitative fluorescence analyses of Aβ plaque area in the hippocampus and cortex, show marked reduction of Aβ plaque burden in mice having increased expression of ACE in microglia and monocyte/MΦ.

FIG. 8 depicts, in accordance with an embodiment herein, reduced levels of cerebral soluble Aβ1-42, using a solid-phase sandwich ELISA, in AD mice havingone or two ACE 10 alleles.

FIG. 9 depicts, in accordance with an embodiment herein, hippocampal images of AD mice with WT and ACE 10/10 phenotype.4G8 identifies Aβ plaques (also indicated by arrows). In AD+ACE10/10mice, ACE is over expressed by microglia and particularly by CD45high monocytes (arrowhead) and MN. The few AP plaques present in AD+ACE10/10 mice are non aggregated. Further, these plaques are often surrounded by MΦ that are internalizing (phagocytizing) Aβ.

FIG. 10 depicts, in accordance with an embodiment herein, analysis of the ability of mice to solve a Barnes maze. 11 month old AD-Tg mice with either wild type ACE expression (AD-Tg:ACEwt/wt), or over expression of ACE in monocytes and macrophages (AD-Tg:ACE 10/10) were tested for their ability to solve a Barnes maze on days 1, 2, 3 and 4. AD-Tg:ACE 10/10 mice were no different from non-AD mice (similar age) and significantly better than AD-Tg:ACEwt/wt mice.

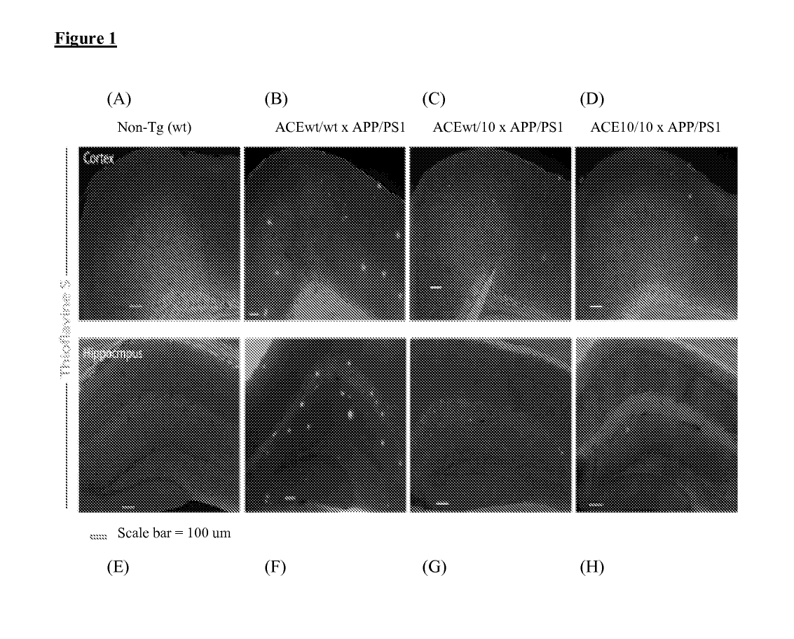

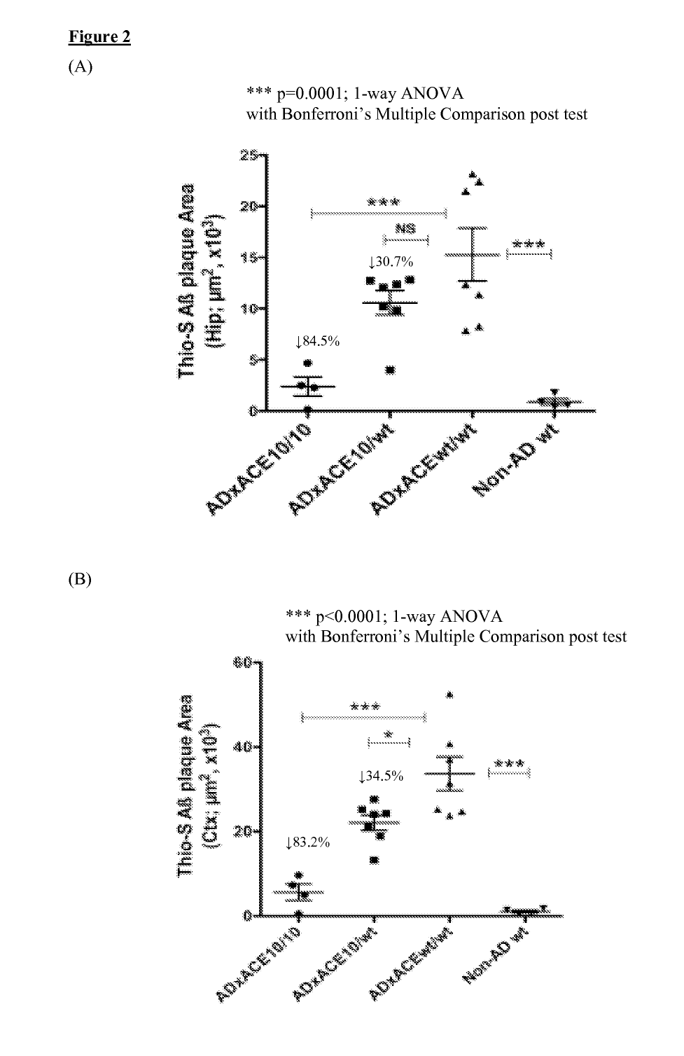

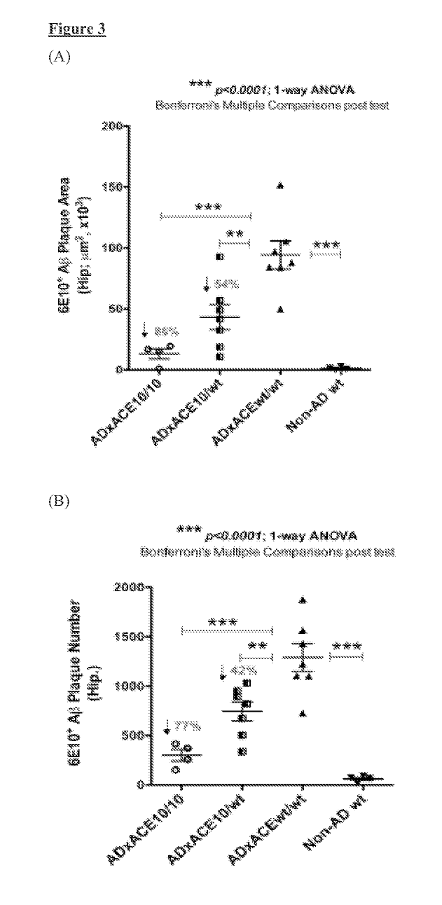

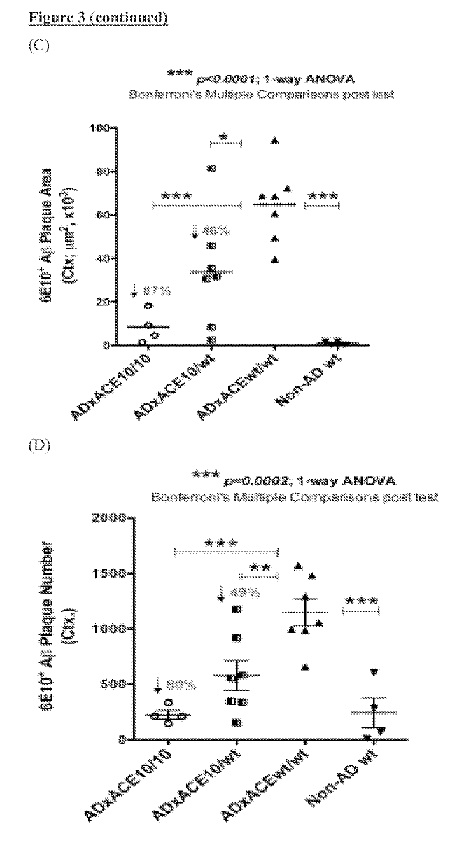

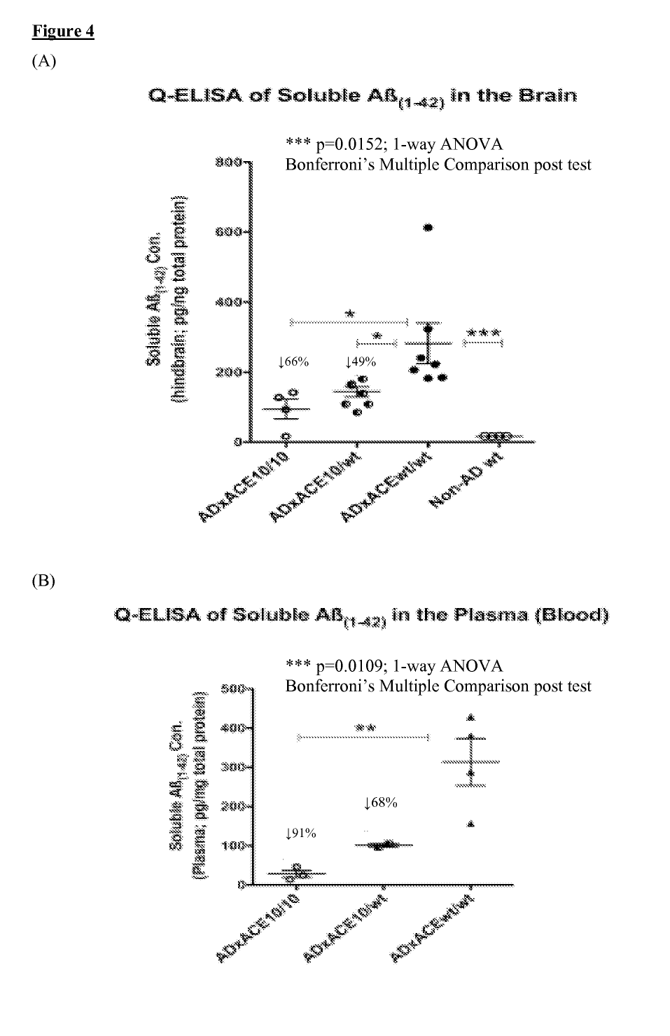

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microhiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor. N Y 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, the term “ACE” means angiotensin converting enzyme, and equivalent peptides. The term “equivalent peptides” means peptides that function in the same manner as ACE insofar as they may cleave amyloid beta plaque.

As used herein, the term “AD” means Alzheimer's disease.

As used herein, “treatment” or “treating” should be understood to include any indicia of success in the treatment, alleviation or amelioration of an injury, pathology or condition. This may include parameters such as abatement, remission, diminishing of symptoms; slowing in the rate of degeneration or decline; making the final point of degeneration less debilitating; improving a patient's physical or mental well-being; or, in some situations, reducing the likelihood of onset of disease.

As used herein, the terms “patient” and “subject” are used interchangeably and refer to human or other mammalian patients and subjects and includes any individual it is desired to examine or treat using the methods of the invention. However, it will be understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g., cats, dogs) and captive wild animals (e.g., foxes, deer, dingoes).

The term “polynucleotide” or “nucleic acid” as used herein designates mRNA, RNA, cRNA, cDNA or DNA. The term typically refers to oligonucleotides greater than 30 nucleotide residues in length.

As used herein, “polypeptide,” “peptide” and “protein” are used interchangeably to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

As disclosed herein, the inventors utilized ACE, an enzyme important in blood pressure control due to ACE mediated conversion of angiotensin I to angiotensin II. ACE is promiscuous in substrate specificity and able to degrade Aβ(1-42), a peptide known be very deleterious in the pathogenesis of Alzheimer's disease. In order to prevent and/or treat the progression of Alzheimer's disease, the inventors cause the overexpression of ACE in the brain, so that the ACE protein will destroy disease causing peptides (proteins) and, by eliminating the underlying causes of Alzheimer's disease, prevent and/or treat this disorder.

As further disclosed herein, the inventors demonstrate that the overexpression of the peptidase ACE in the brain is efficacious in delaying the onset and/or preventing the progression of Alzheimer's disease. Further, the overexpression of any peptidase capable of cleaving amyloid beta plaque peptides and proteins can be efficacious in the treatment of Alzheimer's disease. Further, ACE may be overexpressed in the monocytes and macrophages of a patient with Alzheimer's disease, and these cells may be used to deliver enzyme to the pathogenic peptides/proteins within the brain thus ameliorating this disease. Further, overexpression of ACE by neuroglial cells can also ameliorate this disease. Further, overexpression in monocytes or macrophages of any peptidase capable of destroying amyloidogenic peptides/proteins can also be used to prevent and/or treat Alzheimer's disease.

In one embodiment, the present invention provides a method of treating a mental illness and/or disease in an individual by causing expression of ACE, or a pharmaceutical equivalent, derivative, analog or salt thereof, and/or a peptidase with the capability to inactivate amyloidogenic peptides, in the individual. In another embodiment, the metal illness and/or disease is Alzheimer's disease. In another embodiment, the mental illness and/or disease is dementia. In another embodiment, the mental illness and/or disease is treated by reducing the likelihood of the formation of amyloid plaques. In another embodiment, the mental illness and/or disease is treated by reducing the expansion of amyloid plaques. In another embodiment, the mental illness and/or disease is treated by cleaving Aβ 1-42 peptides. In another embodiment, the mental illness and/or disease is treated by reducing the likelihood of the formation of tau beta fibrils. In another embodiment, the individual is a mouse and/or rat. In another embodiment, the individual is a human. In another embodiment, high expression of ACE is caused by administering a composition comprising monocyte, macrophage, and/or neuroglial cells overexpressing ACE, or a pharmaceutical equivalent, derivative, analog or salt thereof. In another embodiment, the composition is genetically modified to express ACE, or a pharmaceutical equivalent, derivative, analog or salt thereof. In another embodiment, high expression is caused by administering a composition comprising ACE or a pharmaceutical equivalent, derivative, analog or salt thereof, and/or a peptidase with the capability to inactivate amyloidogenic peptides to the individual. In another embodiment, the composition is administered directly to the brain of the individual. In another embodiment, the composition is administered to the individual systemically.

In another embodiment, the present invention provides a method of treatment of a mental illness and/or disease in an individual by genetically modifying bone marrow so that it will increase expression of ACE or a pharmaceutical equivalent, derivative, analog or salt thereof. In another embodiment, bone marrow is taken from the individual, modified in vitro to overexpress ACE, or a pharmaceutical equivalent, derivative, analog or salt thereof, and then followed by reinfusion of the modified marrow back into the individual. In another embodiment, the bone marrow is modified to overexpress ACE, or a pharmaceutical equivalent, derivative, analog or salt thereof by modifying bone marrow-derived monocytes and/or macrophages.

In one embodiment, the present invention provides a composition comprising ACE, or a pharmaceutical equivalent, derivative, analog or salt thereof. In another embodiment, the present invention provides a composition comprising bone marrow-derived cells overexpressing ACE. In another embodiment, the present invention provides a composition comprising one or more cells capable of overexpressing peptidase with the capability to cleave amyloidogenic peptides and/or amyloid beta plaques.

In various embodiments, the present invention provides pharmaceutical compositions including a pharmaceutically acceptable excipient along with a therapeutically effective amount of ACE. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Typical dosages of an effective composition expressing ACE, or ACE composition, can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

The present invention is also directed to a kit to prepare and administer a composition expressing ACE or composition comprising ACE. The kit is useful for practicing the inventive method of treatment of neurological disorders such as Alzheimer's disease. The kit is an assemblage of materials or components, including at least one of the inventive compositions. Thus, in some embodiments the kit contains a composition including ACE, as described above.

The exact nature of the components configured in the inventive kit depends on its intended purpose. For example, some embodiments are configured for the purpose of treating Alzheimer's disease. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to monitor the successful inhibition of amyloid plaques, or administer a therapeutically effective dosage of ACE. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Overexpression of ACE to prevent and treat Alzheimer's disease The inventors utilized angiotensin converting enzyme (ACE), an enzyme important in blood pressure control due to ACE mediated conversion of angiotensin I to angiotensin II. ACE is promiscuous in substrate specificity and able to degrade Aβ (1-42), a peptide known be very deleterious in the pathogenesis of Alzheimer's disease. In order to prevent and/or treat the progression of Alzheimer's disease, the inventors cause the overexpression of ACE in the brain, so that the ACE protein will destroy disease causing peptides (proteins) and, by eliminating the underlying causes of Alzheimer's disease, prevent and/or treat this disorder.

The inventors used a mouse model, called ACE 10/10, in which genetic engineering was used to overexpress ACE in monocytes, macrophages, Kupfer cells, microglia and other tissues utilizing the c-fms promoter. This mouse has previously been characterized. To test the effects of overexpressing ACE, this mouse model was used, as well as the B6.Cg-Tg(APPswe, PSEN1ΔE9)85 Dbo/J mouse, which is a previously published mouse model developing brain plaques, cognitive impairment and other aspects of brain pathology resembling human Alzheimer's disease. The inventors mated these two strains of mice and examined B6.Cg-Tg mice with either WT ACE expression, a single ACE 10/WT gene (heterozygous for ACE) or an animal homozygous for the ACE 10/10 mutation. In essence, the inventors examined the development of Alzheimer's disease in the B6.Cg-Tg mouse as a function of whether ACE was overexpressed in macrophages and microglia. Alzheimer's disease pathology was assessed when mice were seven months old to examine development of Aβ brain deposits/plaques. There was a marked reduction of Aβ plaque numbers and volume in the cortex and hippocampus of mice having either one or two ACE 10/10 genes. In addition, toxic soluble Aβ(1-42) isoforms were reduced in the brains of the ACE 10/10 Alzheimer's mice. In other words, ACE overexpression in macrophages and microglial cells markedly ameliorated the pathology in a mouse model of Alzheimer's disease. Further, the inventors showed that this was associated with a marked reduction in the plasma levels of the soluble Aβ (1-42) peptide, due to the ability of the overexpressed ACE to hydrolyze and destroy this peptide.

The inventors demonstrate that the overexpression of the peptidase ACE in the brain is efficacious in delaying the onset and/or preventing the progression of Alzheimer's disease. Further, the overexpression of any peptidase capable of cleaving the amyloid causing peptides and proteins can be efficacious in the treatment of Alzheimer's disease. Further, ACE may be overexpressed in the monocytes and macrophages of a patient with Alzheimer's disease, and these cells may be used to deliver enzyme to the pathogenic peptides/proteins within the brain thus ameliorating this disease. Further, overexpression of ACE by neuroglial cells can also ameliorate this disease. Further, overexpression in monocytes or macrophages of any peptidase capable of destroying amyloidogenic peptides/proteins can also be used to prevent and/or treat Alzheimer's disease.

### Example 2

### ACE Overexpression Retards AD-Related Pathology

The inventors investigated how the overexpression of a peptidase, ACE, delivered to the brain by myelomonocytic cells (monocytes, macrophages and microglia) affected progression of AD. Their finding is that ACE overexpression retards AD-related pathology. Further, bone marrow transplantation may be used as a means of elevating ACE expression in inflammatory cells to combat AD pathology.

### Example 3

### Aβ Clearance in AD

Pathologically high levels of (P-amyloid (AP) in the brain, both in its soluble and aggregated forms (Aβ plaques), lead to synaptic and neuronal cell loss and progressive cognitive decline.2,3,9-11. Aβ can be cleared across the blood-brain barrier by low-density lipoprotein receptor-related protein-12,13 However, despite this physiological clearance, significant accumulation of Aβ is found in neurons and extracellular deposits in AD patients. According to recent data, sporadic AD is more likely an outcome of inadequate ability to clear Aβ that grows worse with age, rather than Aβ overproduction (as seen in rare familial cases of AD). 14,15 Activated inflammatory cells (microglia, monocytes and macrophages) are critical for the physiological clearance of Aβ. Studies implicate a key role for bone marrow (BM)-derived macrophages (MΦ) in restricting Aβ plaques.4-7,16-28 Data using mouse models of AD demonstrate that boosting levels of blood monocytes increases cerebral infiltration of monocyte-derived MΦ that 1) home to AP lesion sites. 2) restrict Aβ burden, and 3) terminate destructive inflammatory responses.22,29 Shifting monocytes/MΦ phenotype to allow them to better cope with AD pathology, by overexpressing Aβ-degrading enzyme ACE, can present an innovative and superior approach for targeted therapy.

### Example 4

### ACE and AD

ACE is a membrane-bound ectoenzyme that is expressed throughout the body. It has an important role in regulating blood pressure and fluid homeostasis mainly via production of the vasoactive peptide angiotensin II.30 Angiotensin I and bradykinin are well-known substrates of ACE, but this enzyme is promiscuous and also cleaves substance P, β-endorphins, AP and many other peptides. Because of this variety of substrates. ACE affects many physiological processes.31,32 In vitro studies demonstrated the ability of ACE to cleave Aβ1-40 and Aβ1-42,33-35 and convert the deleterious Aβ1-42 to the shorter, more benign Aβ1-40 isoforms.1,36

### Example 5

### Creation of ACE10/10 Mice

Macrophages (Mc) participate in virtually all aspects of the immune response and one of the products made by activated MΦ is ACE.49 To test the role of MC-ACE in inflammation, the inventors created a mouse strain, in which ACE is highly overexpressed by myelomonocytic lineage cells, such as monocytes, MΦ and microglia.8 Specifically, the inventors used targeted homologous recombination in ES cells to insert an artificial c-fms promoter to control ACE expression (a 6.7 kb c-fms promoter cassette contained both enhancer and core promoter sequences sufficient to initiate transcription). In these mice endogenous ACE expression is removed from its normal localization of expression and redirected to myelomonocytic cells. In essence, the strategy eliminated any effects of the endogenous ACE promoter, such that ACE expression is now completely controlled by tissue recognition of the c-fms promoter cassette.

The ACE genotypes are wild-type (ACEwt/wt), heterozygous (ACE10/wt) and homozygous for the mutant allele (ACE10/10). ACE10/10 mice have normal body size and organ weights. They have a typical lifespan with no basal physiological defects. They also have blood pressures indistinguishable from WT mice. The expression of ACE in ACE10/10 mice was studied using ACE enzymatic activity assays, Western blot analysis, immunohistochemistry and FACS analyses. These studies showed that ACE10/10 mice lack ACE expression by vascular endothelium. In contrast, monocytes, MΦ and microglia have far more ACE protein (about 14-fold) than the equivalent tissues in WT mice. In summary, a variety of studies showed that ACE10/10 mice have constitutive ACE expression targeted to myelomonocytic lineage cells.

### Example 6

### ACE10/10 and Immunity

An indication that ACE10/10 mice were different from WT mice came when the inventors challenged the mice with B16 melanoma cells.8 This is an aggressive cell line and implanted tumor cells will grow into a large tumor nodule in most mouse strains. However, the inventors found a profound and consistent difference between WT and ACE10/10 mice (both on C57BL/6 background); tumors were much smaller in ACE10/10. Increased resistance to melanoma in ACE10/10 was associated with increased monocytes and MΦ within the tumor, increased phagocytosis of tumor cells, increased MΦ expression of pro-inflammatory cytokines and increased numbers of anti-tumor CD8+ T cells.8 The phenotype of increased resistance to melanoma was transferable by BM transplantation and reverted by ACE inhibitors.

The inventors also tested the resistance of ACE10/10 mice to a variety of infectious agents, including bacteria and viruses.50 ACE10/10 mice consistently responded to these immune challenges with an increased immune response and substantially decreased disease. ACE10/10 mice show no evidence of autoimmunity. Additional characterization of MΦ function in ACE10/10 mice led to the realization that the innate immune cells in this model respond to immune challenge with a tilt towards the pro-inflammatory phenotype referred to as MΦ ‘classical’ activation.51.52 Such MΦ have an enhanced ability to phagocytize and destroy foreign proteins and organisms. The combination of this MΦ phenotype and the enhanced expression of surface ACE, a peptidase known to be capable of destroying amyloidogenic peptides, led to the finding that expression of the ACE10/10 phenotype in a mouse genetically predisposed to AD would lead to reduced disease pathogenesis. Specifically, ACE peptidase, delivered to the brain at the right time and place, will cleave pathogenic Aβ, facilitating its efficient clearance by monocytes/MΦ, thereby safely eliminating the underlying cause of AD.

### Example 7

### Cross of ACE10/10 Mice with AD-Tg Mice

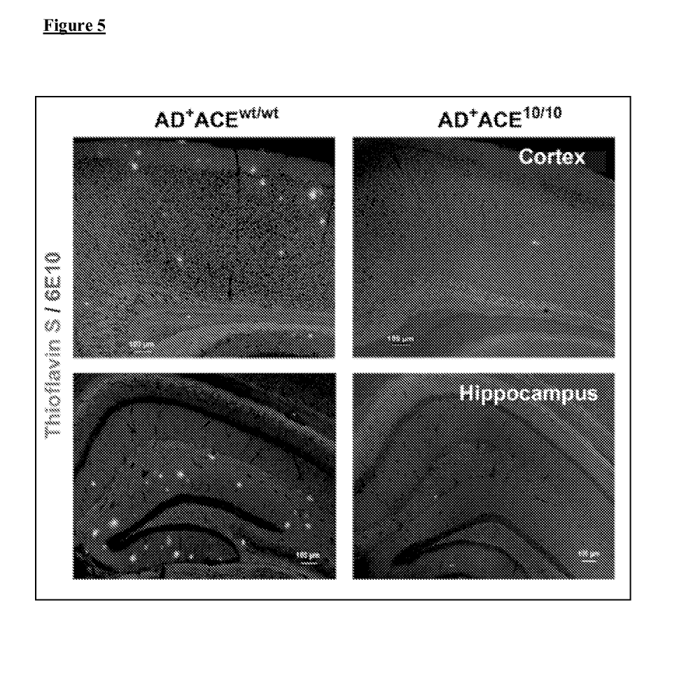

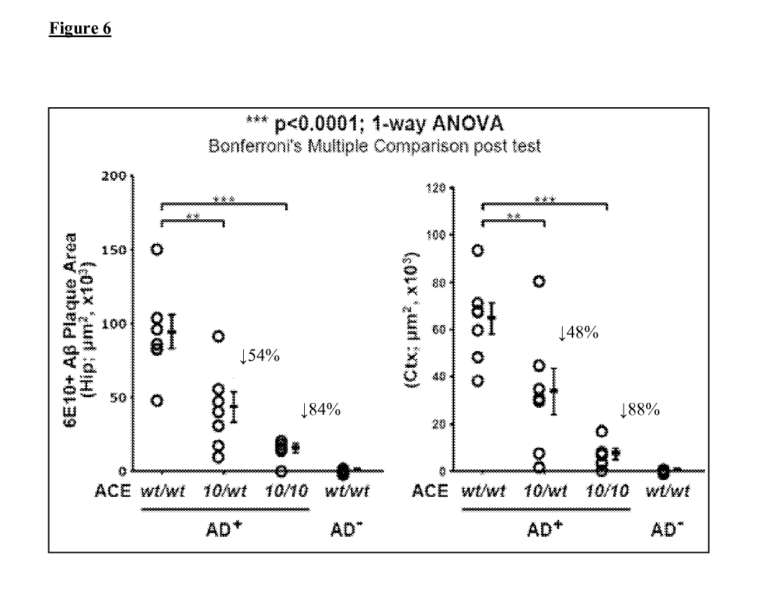

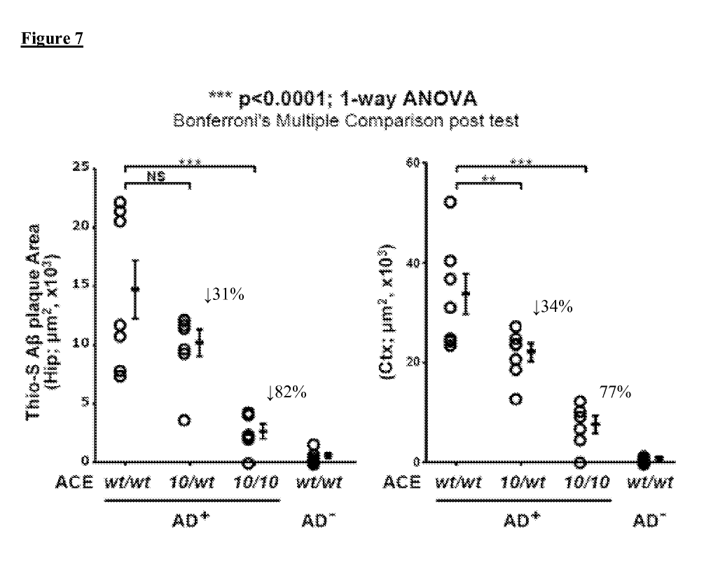

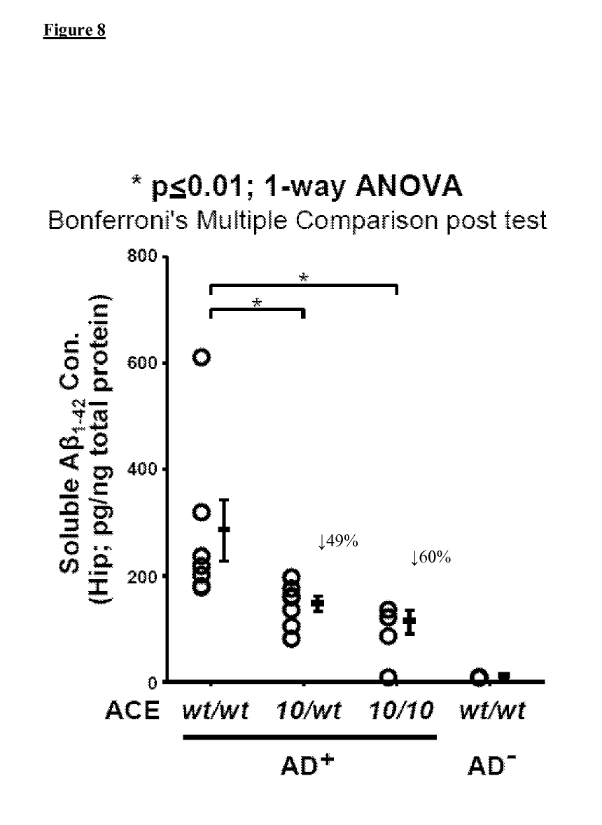

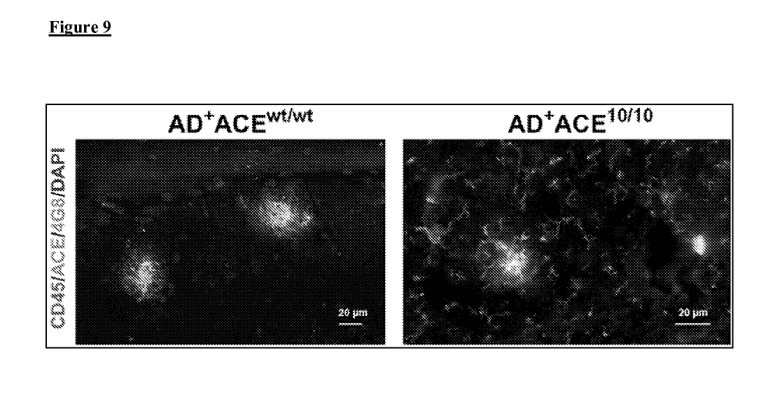

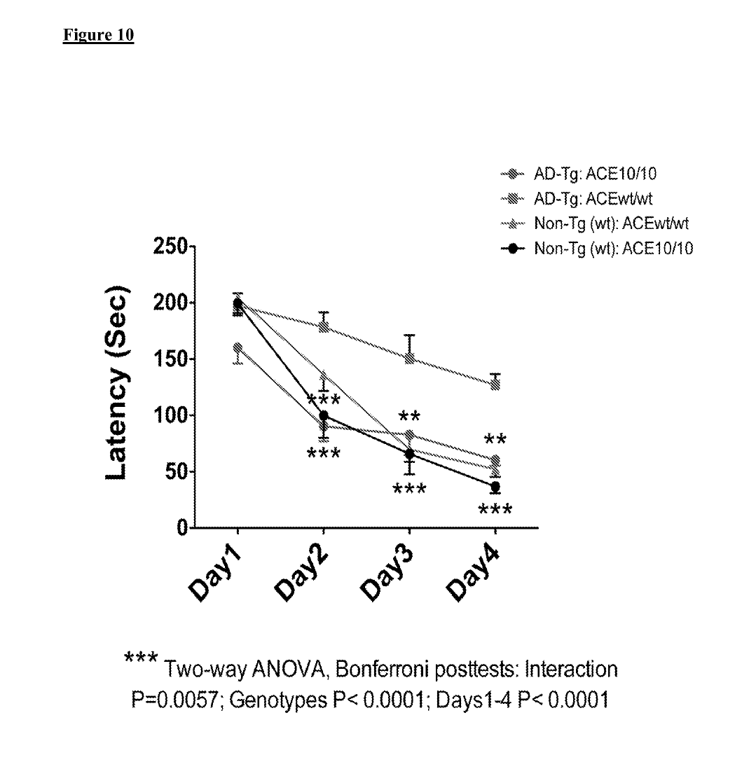

To examine the effects of ACE overexpression in AD, the inventors crossed ACE10/10 mice with a double-transgenic mouse model of AD [strain B6.Cg Tg(APPSWE, PSEN1ΔE9) 85 Dbo/J; referred to here as AD-Tg mice]. This is a well-established mouse model for AD, harboring familial AD mutations: chimeric mouse/human APP (APPSWE) and human presenilin 1 (PS1ΔE9) genes whose expression is directed to CNS neurons by the prion protein (PrP) promoter.53 As a result. AD-Tg mice over-produce amyloidogenic AP peptides in the brain, develop AP plaques with age, exhibit increased cerebral amyloid angiopathy and show progressive cognitive impairments with other neuropathological features mimicking human AD.54-63 The inventors evaluated the development of AD in AD-Tg mice as a function of ACE expression. The resulting data indicated a huge reduction (over 80%) of cerebral AP plaque burden in AD-Tg mice having two ACE 10 alleles. Moreover, levels of soluble neurotoxic AP1-42 peptides, directly associated with AD cognitive decline, substantially decreased in the brains and plasma of the crossed mice.64,65 Specifically, the inventors studied AD-Tg mice with WT ACE expression (AD+ACEwt/vt; n=7), a single ACE 10 allele (AD+ACE10/wt; n=7), or two ACE. alleles (AD+ACE10/10; n=6). The inventors also studied control mice that were non-AD and WT for ACE (AD-ACEwt/wt; n=6). Pathology was assessed at 7 months, the age at which AD-Tg mice exhibit AD-like symptoms and neuropathology.54,56 To measure cerebral AD burden, the inventors used Thioflavine-S (Thio-S) labeling of fibrillar/mature plaques and immunolabeling with 6E10 mAb (recognizing aa residue 1-16 of human AD). Representative fluorescent micrographs of cortical (FIG. 5, top) and hippocampal (FIG. 5, bottom) Aβ plaques demonstrated almost a complete elimination of cerebral Aβ plaques in AD+ACE10/10 mice. Quantitative analysis of 6E10-immunoreactive AD plaque area in the hippocampus and cortex showed a reduction of about 50% in mice having one ACE 10 allele (AD+ACE10/wt) and above 80% reduction in mice having two ACE 10 alleles (AD+ACE10/10) (FIG. 6; p<0.0001). A similar quantitative analysis of Thio-S-positive Aβ plaque area in the hippocampus and cortex also demonstrated a very large reduction (˜80%) in mice carrying two ACE 10 alleles (FIG. 7; p<0.0001). Plaque numbers, whether individually assessed with 6E10 or Thio-S, showed reductions equivalent to the overall reductions of plaque area. Quantitative ELISA measurements of soluble Aβ1-42 levels in the hippocampus demonstrated a significant reduction of about 50% and 60% in AD+ACE10/wt or AD+ACE10/10, respectively (FIG. 8; p≦0.01). The decrease in toxic Aβ1-42 isoform correlated well with reduced plaque size and number. ELISA assessments of soluble AD1-42 levels in the plasma indicated an even higher reduction: about 60% reduction in AD+ACE10/wt and 80% reduction in AD+ACE10/10, as compared to the AD+ACEwt/wt mice (p≦0.01). Please note that AD+ACE10/10 mice have plasma levels of soluble ACE that are not significantly different from those of wild-type mice.8 Thus, reduction of Aβ pathology correlated with ACE expression by myelomonocytic cells. Immunolabeling in the brains of AD+ACE10/10 mice showed high levels of ACE expression by monocytes and MΦ (CD45high in red); ACE expression by microglia was also elevated, but was less than by monocytic cells (FIG. 9, right image). Upon activation, microglia and MΦ share many phenotypical markers and can exert similar activities making it hard to distinguish by classical histology. The inventors used the hematopoietic CD45 marker to distinguish between microglia and infiltrating monocytes/MΦ: the latter show intense CD45 staining but not the resident microglia, even in their activated state.6,68-71 ACE expression was especially strong in areas surrounding Aβ plaques (anti-human Aβ 4G8 mAb) where MΦ were often present and especially in monocytes arriving from blood vessels (FIG. 9; arrowhead). In contrast, ACE expression is very low in AD mice with wt ACE expression (FIG. 9, left image; AD+ACEwt/wt). In the 7-month-old AD+ACEwt/wt mice brains, multiple large and aggregated Aβ plaques were frequently found, whereas, only small residual and diffused plaques were detected in the AD+ACE10/wt and AD+ACE10/10 mice (FIG. 9). The CD45high-M in AD+ACE10/10 mice appear directly involved in AP clearance by phagocytosis (FIG. 9; white spot inside MΦ associated with the plaque). Overall, these preliminary data show that ACE overexpression in cells of myelo-monocytic origin can markedly ameliorate AD-like neuropathology. This dramatic decrease in AP burden is due to 1) the ability of ACE to hydrolyze and destroy A peptide, and 2) the enhanced effectiveness of ACE overexpressing MΦ to clear A from the brain and blood. Possibly, their increased immunological activity also supports tissue repair processes including neurotrophic effects. The work supports that overexpression of the peptidase ACE in the brain by innate immune cells is efficient in delaying/preventing and curing AD.

### Example 8

### Bone Marrow Transplantation

In AD mouse models, BM-derived monocyte/MΦ play a pivotal role in restricting Aβ plaques, possibly also inducing immunoregulation and tissue repair.4-7,28-29 BM transplantation into AD-Tg (AD+ACEwt/wt) mice of BM from GFP-AD-ACE10/10, or GFP-AD-ACEwt/wt, or syngeneic AD+ACEwt/wt mice may be performed. In short, 2- to 3-month-old AD+ACEwt/wt mice may be lethally irradiated with 1100 rad and then immediately reconstituted with 2×106 donor marrow cells (isolated from 2-month-old GFP-expressing donor mice). After 8 weeks of marrow repopulation, engraftment will be assessed by FACS analysis of peripheral blood for GFP expression. ACE10/10 mice expressing GFP prepared and on a C57BL/6 background. A limitation is that irradiation of the brain may significantly alter the rate of infiltrating immune cells and the progression of AD. To eliminate this, protect the brains from irradiation with extensive lead shielding.

### Example 9

### Adoptive Transfer of Monocytes

Another therapeutic approach is to enrich the peripheral blood of AD-Tg mice with ACE10/10 CD115/c-fms+-BM monocytes. The inventors' data indicate that transfusing inflammatory (CD115+) WT monocytes into the blood of 5- and 7-months old AD-Tg mice significantly increases monocyte infiltration to cerebral Aβ lesions and reduces Aβ plaque burden.29 This subset of inflammatory monocytes was reported to directly enter inflamed tissues and possibly replace microglia under inflammatory conditions.72-78 AD+ACEwt/wt mice may receive monthly blood injections of GFP-ACE10/10 or GFP-ACEwt/wt monocytes for a treatment period of 3 months, starting from the symptomatic age of 7 months. BM cells may be harvested, and enriched for mononuclear cells on a Ficoll density gradient. CD115+ monocytes isolated through MACS enrichment using biotinylated anti-CD 115 antibodies and streptavidin-coupled magnetic beads (Miltenyi Biotec) may be injected (2×106 cells) intravenously once a month to ensure long-term elevated levels of transfused monocytes. This procedure will enrich this subset of monocytes in the blood by 3-fold since their total number in the mouse blood is approximately 106 cells.73,74,76

### Example 10

### Characterization and Data Analysis

Characterization of isolated BM-derived monocytes may be performed by FACS as previously described.79,80 The inventors demonstrated that WT monocytes injected into the peripheral blood have the ability to infiltrate AD brains and attenuate AD progression. ACE10/10 monocytes may be more effective in fighting AD. The chimeric mice may be studied for their memory and learning behavior, as well as brain pathology at 10 month of age. In addition, the recruitment of monocyte-derive MΦ in the brain may be measured as follows. The analysis of GFP-labeled monocytes/MΦ within brains may include: 1. Quantitative IHC counts of GFP-labeled and Iba-1+/CD45high (or CD11b+/CD45high) MΦ at cerebral Aβ plaque sites (using ImageJ Software). Also, may use well-established blinded manual subfield counting methods (using MBF Bioscience Stereology system) 2. Flow cytometry of GFP/CD11b/CD45-triple positive cells in the brain.

Data Quantification: For microscopic analysis, a Karl Zeiss fluorescence microscope (Imager, Z1, ApoTome and MBF-equipped) is used. Coronal sections of the brain are analyzed. Numbers, area, and intensity of Aβ deposits, Glial fibrillary acidic protein cells (GFAP+, an astrocytes marker) and GFP infiltrating cells, are determined automatically with NIH ImageJ software or by a MBF stereological system. Alternatively, manual quantification of cells is done using Image J and/or determined by FACS.

Statistical analysis: Normally distributed data, resulting from IHC, ELISA, WB or FACS analyses, are expressed as means±SEM. One-way analysis of variance (ANOVA) with Bonferroni (or other suitable) post-hoc test is used for multiple comparisons (three or more means). For more advanced statistical analyses, such as for behavioral tests, repeated-measures two-way ANOVA (mixed model) is used. On-campus statistical core facility is available for further specialized assistance. The threshold for statistical significance is set at 0.05. For most behavioral testing, a power of 80% is obtained with 8-10 animals per group. For stereology or other semi or fully quantitative analysis (i.e. q-IHC, WB, ELISA), 6-8 mice per group have sufficient power to detect significance. Two-group comparison is analyzed by unpaired Student's t test.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context.

The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

1. Zou K, Maeda T, Watanabe A, Liu J, Liu S, Oba R, Satoh Y, Komano H, Michikawa M: Abeta42-to-Abeta40- and angiotensin-converting activities in different domains of angiotensin-converting enzyme, J Biol Chem 2009, 284:31914-31920
2. Hardy J, Selkoe DJ: The amyloid hypothesis of Alzheimer's disease: progress and problems on the road to therapeutics, Science 2002, 297:353-356
3. Hardy J A, Higgins G A: Alzheimer's disease: the amyloid cascade hypothesis, Science 1992, 256:184-185
4. Butovsky O, Kunis G, Koronyo-Hamaoui M, Schwartz M: Selective ablation of bone marrow-derived dendritic cells increases amyloid plaques in a mouse Alzheimer's disease model, Eur J Neurosci 2007, 26:413-416
5. El Khoury J, Toft M, Hickman S E, Means T K, Terada K, Geula C, Luster A D: Ccr2 deficiency impairs microglial accumulation and accelerates progression of Alzheimer-like disease, Nat Med 2007, 13:432-438
6. Koronyo-Hamaoui M, Ko M K, Koronyo Y. Azoulay D, Seksenyan A, Kunis G, Pham M. Bakhsheshian J, Rogeri P, Black K L, Farkas D L, Schwartz M: Attenuation of AD-like neuropathology by harnessing peripheral immune cells: local elevation of IL-10 and MMP-9, J Neurochem 2009, 111:1409-1424
7. Simard A R, Soulet D, Gowing G, Julien J P, Rivest S: Bone marrow-derived microglia play a critical role in restricting senile plaque formation in Alzheimer's disease, Neuron 2006, 49:489-502
8. Shen X Z, Li P, Weiss D, Fuchs S, Xiao H D, Adams J A, Williams I R, Capecchi M R, Taylor W R, Bernstein K E: Mice with enhanced macrophage angiotensin-converting enzyme are resistant to melanoma, Am J Pathol 2007, 170:2122-2134
9. Glenner G G, Wong C W, Quaranta V, Eanes E D: The amyloid deposits in Alzheimer's disease: their nature and pathogenesis, Appl Pathol 1984, 2:357-369
10. Price D L, Sisodia S S: Cellular and molecular biology of Alzheimer's disease and animal models, Annu Rev Med 1994, 45:435-446
11. Selkoe D J: Alzheimer's disease. In the beginning, Nature 1991, 354:432-433
12. Yamada K, Hashimoto T, Yabuki C, Nagae Y, Tachikawa M, Strickland D K. Liu Q, Bu G, Basak J M, Holtzman D M, Ohtsuki S, Terasaki T, Iwatsubo T: The low density lipoprotein receptor-related protein I mediates uptake of amyloid beta peptides in an in vitro model of the blood-brain barrier cells, J Biol Chem 2008, 283:34554-34562
13. Zlokovic B V. Deane R, Sagare A P, Bell R D, Winkler E A: Low-density lipoprotein receptor-related protein-1: a serial clearance homeostatic mechanism controlling Alzheimer's amyloid beta-peptide elimination from the brain, J Neurochem 115:1077-1089
14. Kirkitadze M D, Kowalska A: Molecular mechanisms initiating amyloid beta-fibril formation in Alzheimer's disease. Acta Biochim Pol 2005, 52:417-423
15. Saido TC: Alzheimer's disease as proteolytic disorders: anabolism and catabolism of beta-amyloid, Neurobiol Aging 1998, 19:S69-75
16. Butovsky O, Koronyo-Hamaoui M. Kunis G. Ophir E. Landa G, Cohen H, Schwartz M: Glatiramer acetate fights against Alzheimer's disease by inducing dendritic-like microglia expressing insulin-like growth factor 1, Proc Natl Acad Sci USA 2006, 103:11784-11789
17. Cameron B, Landreth G E: Inflammation, microglia, and Alzheimer's disease, Neurobiol Dis 37:503-509
18. Fiala M, Cribbs D H, Rosenthal M, Bernard G: Phagocytosis of amyloid-beta and inflammation: two faces of innate immunity in Alzheimer's disease, J Alzheimers Dis 2007, 11:457-463
19. Glezer I, Simard A R, Rivest S: Neuroprotective role of the innate immune system by microglia, Neuroscience 2007, 147:867-883
20. Hanisch U K, Kettenmann H: Microglia: active sensor and versatile effector cells in the normal and pathologic brain, Nat Neurosci 2007, 10:1387-1394
21. Hickman S E, El Khoury J: Mechanisms of mononuclear phagocyte recruitment in Alzheimer's disease, CNS Neurol Disord Drug Targets 9:168-173
22. Lebson L, Nash K, Kamath S, Herber D, Carty N, Lee D C, Li Q, Szekeres K. Jinwal U, Koren J, Dickey C A, Gottschall P E, Morgan D. Gordon M N: Trafficking CD11b-positive blood cells deliver therapeutic genes to the brain of amyloid-depositing transgenic mice, J Neurosci 30:9651-9658
23. Malm T M. Koistinaho M, Parepalo M. Vatanen T, Ooka A, Karlsson S, Koistinaho J: Bone-marrow-derived cells contribute to the recruitment of microglial cells in response to beta-amyloid deposition in APP/PS1 double transgenic Alzheimer mice, Neurobiol Dis 2005, 18:134-142
24. Paresce D M. Ghosh R N, Maxfield F R: Microglial cells internalize aggregates of the Alzheimer's disease amyloid beta-protein via a scavenger receptor, Neuron 1996, 17:553-565
25. Takata K, Kitamura Y, Yanagisawa D. Morikawa S, Morita M, Inubushi T, Tsuchiya D. Chishiro S, Saeki M, Taniguchi T, Shimohama S. Tooyama I: Microglial transplantation increases amyloid-beta clearance in Alzheimer model rats, FEBS Lett 2007, 581:475-478
26. Town T, Laouar Y, Pittenger C, Mori T, Szekely C A, Tan J, Duman R S, Flavell R A: Blocking TGF-beta-Smad2/3 innate immune signaling mitigates Alzheimer-like pathology. Nat Med 2008, 14:681-687
27. Wyss-Coray T. Lin C. Yan F. Yu G Q, Rohde M, McConlogue L, Masliah E, Mucke L: TGF-beta1 promotes microglial amyloid-beta clearance and reduces plaque burden in transgenic mice, Nat Med 2001, 7:612-618
28. Richard K L, Filali M, Prefontaine P, Rivest S: Toll-like receptor 2 acts as a natural innate immune receptor to clear amyloid beta 1-42 and delay the cognitive decline in a mouse model of Alzheimer's disease, J Neurosci 2008, 28:5784-5793
29. Salumbides B, KORONYO Y, PHAM M, MOYSEYEV M, LJUBIMOV V, LIAM M, Black K L, IRVIN D K, SCHWARTZ M, KORONYO-HAMAOUI M: Immune-based combination therapy reduces Alzheimer's Disease pathology in preclinical models, J Neurosci 2010, Suppl Socienty for Neuroscience:abstract 556
30. Corvol P, Williams T A, Soubrier F: Peptidyl dipeptidase A: angiotensin 1-converting enzyme. Methods Enzymol 1995, 248:283-305
31. Metzger R, Bohle R M, Chumachenko P, Danilov S M, Franke F E: CD143 in the development of atherosclerosis, Atherosclerosis 2000, 150:21-31
32. Metzger R, Bohle R M, Pauls K, Eichner G, Alhenc-Gelas F, Danilov S M, Franke FE: Angiotensin-converting enzyme in non-neoplastic kidney diseases, Kidney Int 1999. 56:1442-1454
33. Hemming M L, Selkoe D J: Amyloid beta-protein is degraded by cellular angiotensin-converting enzyme (ACE) and elevated by an ACE inhibitor, J Biol Chem 2005, 280:37644-37650
34. Hu J, Igarashi A, Kamata M, Nakagawa H: Angiotensin-converting enzyme degrades Alzheimer amyloid beta-peptide (A beta); retards A beta aggregation, deposition, fibril formation; and inhibits cytotoxicity. J Biol Chem 2001, 276:47863-47868
35. Oba R, Igarashi A, Kamata M, Nagata K, Takano S, Nakagawa H: The N-terminal active centre of human angiotensin-converting enzyme degrades Alzheimer amyloid beta-peptide, Eur J Neurosci 2005, 21:733-740
36. Zou K, Yamaguchi H, Akatsu H. Sakamoto T, Ko M. Mizoguchi K, Gong J S, Yu W, Yamamoto T, Kosaka K, Yanagisawa K, Michikawa M: Angiotensin-converting enzyme converts amyloid beta-protein 1-42 (Abeta(1-42)) to Abeta(1-40), and its inhibition enhances brain Abeta deposition, J Neurosci 2007, 27:8628-8635
37. Miners J S. Ashby E, Van Helmond Z, Chalmers K A. Palmer L E, Love S, Kehoe P G: Angiotensin-converting enzyme (ACE) levels and activity in Alzheimer's disease, and relationship of perivascular ACE-1 to cerebral amyloid angiopathy, Neuropathol Appl Neurobiol 2008, 34:181-193
38. Savaskan E. Hock C, Olivieri G, Bruttel S. Rosenberg C, Hulette C, Muller-Spahn F: Cortical alterations of angiotensin converting enzyme, angiotensin 11 and AT1 receptor in Alzheimer's dementia, Neurobiol Aging 2001, 22:541-546
39. Elkins J S, Douglas V C, Johnston S C: Alzheimer disease risk and genetic variation in ACE: a meta-analysis, Neurology 2004, 62:363-368
40. Kehoe P G, Katzov H, Andreasen N, Gatz M, Wilcock G K, Cairns N J, Palmgren J, de Faire U, Brookes A J, Pedersen N L, Blennow K, Prince J A: Common variants of ACE contribute to variable age-at-onset of Alzheimer's disease, Hum Genet 2004, 114:478-483
41. Kehoe P G, Katzov H. Feuk L, Bennet A M, Johansson B, Wiman B, de Faire U, Cairns N J, Wilcock G K, Brookes A J, Blennow K, Prince J A: Haplotypes extending across ACE are associated with Alzheimer's disease. Hum Mol Genet 2003, 12:859-867
42. Kehoe P G, Russ C, Mcllory S. Williams H, Holmans P, Holmes C, Liolitsa D, Vahidassr D, Powell J, McGleenon B, Liddell M, Plomin R, Dynan K, Williams N, Neal J, Cairns N J, Wilcock G, Passmore P,
Lovestone S, Williams J, Owen MJ: Variation in DCP1, encoding ACE, is associated with susceptibility to Alzheimer disease, Nat Genet 1999, 21:71-72
43. Lehmann D J, Cortina-Borja M, Warden D R, Smith A D, Sleegers K, Prince J A, van Duijn C M, Kehoe P G: Large meta-analysis establishes the ACE insertion-deletion polymorphism as a marker of Alzheimer's disease, Am J Epidemiol 2005, 162:305-317
44. Narain Y, Yip A, Murphy T, Brayne C, Easton D, Evans J G, Xuereb J, Cairns N, Esiri M M. Furlong R A, Rubinsztein D C: The ACE gene and Alzheimer's disease susceptibility, J Med Genet 2000, 37:695-697
45. Kolsch H, Jessen F, Freymann N. Kreis M, Hentschel F. Maier W, Heun R: ACE I/D polymorphism is a risk factor of Alzheimer's disease but not of vascular dementia, Neurosci Lett 2005, 377:37-39
46. Eckman E A, Adams S K, Troendle F J, Stodola B A, Kahn M A, Fauq A H, Xiao H D. Bernstein K E, Eckman C B: Regulation of steady-state beta-amyloid levels in the brain by neprilysin and endothelin-converting enzyme but not angiotensin-converting enzyme, J Biol Chem 2006, 281:30471-30478
47. Hemming M L, Selkoe D J. Farris W: Effects of prolonged angiotensin-converting enzyme inhibitor treatment on amyloid beta-protein metabolism in mouse models of Alzheimer disease, Neurobiol Dis 2007, 26:273-281
48. Lendon C L, Thaker U, Harris J M, McDonagh A M, Lambert J C, Chartier-Harlin M C, Iwatsubo T, Pickering-Brown S M, Mann D M: The angiotensin 1-converting enzyme insertion (I)/deletion (D) polymorphism does not influence the extent ofamyloid or tau pathology in patients with sporadic Alzheimer's disease, Neurosci Lett 2002, 328:314-318
49. Saijonmaa O, Nyman T, Fyhrquist F: Atorvastatin inhibits angiotensin-converting enzyme induction in differentiating human macrophages, Am J Physiol Heart Circ Physiol 2007, 292:H1917-1921
50. Okwan-Duodu D. Datta V, Shen X Z, Goodridge H S, Bernstein E A. Fuchs S, Liu G Y, Bernstein K E: Angiotensin-converting enzyme overexpression in mouse myelomonocytic cells augments resistance to Listeria and methicillin-resistant Staphylococcus aureus, J Biol Chem 285:39051-39060
51. Gordon S. Taylor P R: Monocyte and macrophage heterogeneity, Nat Rev Immunol 2005, 5:953-964
52. Mantovani A, Sica A, Locati M: Macrophage polarization comes of age, Immunity 2005, 23:344-346
53. Jankowsky J L. Slunt H H, Ratovitski T, Jenkins N A, Copeland N G. Borchelt D R: Co-expression of multiple transgenes in mouse CNS: a comparison of strategies, Biomol Eng 2001, 17:157-165
54. Garcia-Alloza M, Robbins E M, Zhang-Nunes S X, Purcell S M, Betensky R A, Raju S. Prada C, Greenberg S M, Bacskai B J, Frosch M P: Characterization of amyloid deposition in the APPswe/PS1dE9 mouse model of Alzheimer disease, Neurobiol Dis 2006, 24:516-524
55. Goto Y, Niidome T, Hongo H, Akaike A, Kihara T, Sugimoto H: Impaired muscarinic regulation of excitatory synaptic transmission in the APPswe/PS1dE9 mouse model of Alzheimer's disease. Eur J Pharmacol 2008, 583:84-91
56. Jankowsky J L, Fadale D J, Anderson J, Xu G M, Gonzales V, Jenkins N A, Copeland N G, Lee M K, Younkin L H, Wagner S L, Younkin S G, Borchelt D R: Mutant presenilins specifically elevate the levels of the 42 residue beta-amyloid peptide in vivo: evidence for augmentation of a 42-specific gamma secretase, Hum Mol Genet 2004, 13:159-170
57. Machova E, Jakubik J. Michal P, Oksman M, livonen H. Tanila H. Dolezal V: Impairment of muscarinic transmission in transgenic APPswe/PS1dE9 mice, Neurobiol Aging 2008, 29:368-378
58. Niidome T, Taniuchi N, Akaike A, Kihara T, Sugimoto H: Differential regulation of neurogenesis in two neurogenic regions of APPswe/PS1dE9 transgenic mice, Neuroreport 2008, 19:1361-1364
59. O'Leary T P, Brown R E: Visuo-spatial learning and memory deficits on the Barnes maze in the 16-month-old APPswe/PS1dE9 mouse model of Alzheimer's disease, Behav Brain Res 2009, 201:120-127
60. Ramirez-Lugo L, Jensen M S, Soderman A, West M J: Deficits in aversive but not in safe taste memory in the APPswe/PS1dE9 mice. J Alzheimers Dis 2009, 18:281-293
61. Ruan L. Kang Z, Pei G, Le Y: Amyloid deposition and inflammation in APPswe/PSIdE9 mouse model of Alzheimer's disease, Curr Alzheimer Res 2009, 6:531-540
62. Savonenko A, Xu G M, Melnikova T, Morton J L, Gonzales V, Wong M P, Price D L, Tang F. Markowska A L. Borchelt D R: Episodic-like memory deficits in the APPswe/PS1dE9 mouse model of Alzheimer's disease: relationships to beta-amyloid deposition and neurotransmitter abnormalities, Neurobiol Dis 2005, 18:602-617
63. Taniuchi N, Niidome T, Goto Y, Akaike A, Kihara T, Sugimoto H: Decreased proliferation of hippocampal progenitor cells in APPswe/PS1dE9 transgenic mice, Neuroreport 2007, 18:1801-1805
64. Selkoe D J: Soluble oligomers of the amyloid beta-protein impair synaptic plasticity and behavior, Behav Brain Res 2008, 192:106-113
65. Shankar G M, Li S, Mehta T H. Garcia-Munoz A, Shepardson N E, Smith I, Brett F M, Farrell M A, Rowan M J, Lemere C A, Regan C M, Walsh D M, Sabatini B L, Selkoe D J: Amyloid-beta protein dimers isolated directly from Alzheimer's brains impair synaptic plasticity and memory, Nat Med 2008, 14:837-842
66. Soulet D, Rivest S: Bone-marrow-derived microglia: myth or reality?, Curr Opin Pharmacol 2008, 8:508-518
67. Reiserer R S, Harrison F E, Syverud D C, McDonald M P: Impaired spatial learning in the APPSwe+PSEN1DeltaE9 bigenic mouse model of Alzheimer's disease, Genes Brain Behav 2007, 6:54-65
68. Ford A L, Goodsall A L, Hickey W F, Sedgwick J D: Normal adult ramified microglia separated from other central nervous system macrophages by flow cytometric sorting. Phenotypic differences defined and direct ex vivo antigen presentation to myelin basic protein-reactive CD4+ T cells compared, J Immunol 1995, 154:4309-4321
69. Juedes A E, Ruddle N H: Resident and infiltrating central nervous system APCs regulate the emergence and resolution of experimental autoimmune encephalomyelitis, J Immunol 2001, 166:5168-5175
70. Sedgwick J D, Schwender S, Imrich H, Dorries R, Butcher G W, ter Meulen V: Isolation and direct characterization of resident microglial cells from the normal and inflamed central nervous system, Proc Natl Acad Sci USA 1991, 88:7438-7442
71. Stoll G, Jander S: The role of microglia and macrophages in the pathophysiology of the CNS, Prog Neurobiol 1999, 58:233-247
72. Auffray C, Fogg D K, Narni-Mancinelli E, Senechal B, Trouillet C, Saederup N, Leemput J, Bigot K, Campisi L, Abitbol M, Molina T, Charo I, Hume D A, Cumano A, Lauvau G, Geissmann F: CX3CR1+CD115+CD135+ common macrophage/DC precursors and the role of CX3CR1 in their response to inflammation, J Exp Med 2009, 206:595-606
73. Auffray C, Sieweke M H, Geissmann F: Blood monocytes: development, heterogeneity, and relationship with dendritic cells, Annu Rev Immunol 2009, 27:669-692
74. Geissmann F. Auffray C, Palframan R, Wirrig C. Ciocca A, Campisi L, Narni-Mancinelli E, Lauvau G: Blood monocytes: distinct subsets, how they relate to dendritic cells, and their possible roles in the regulation of T-cell responses, Immunol Cell Biol 2008, 86:398-408
75. Huang B. Pan P Y, Li Q, Sato A I, Levy D E, Bromberg J, Divino C M, Chen S H: Gr-1+CD115+ immature myeloid suppressor cells mediate the development of tumor-induced T regulatory cells and T-cell anergy in tumor-bearing host, Cancer Res 2006, 66:1123-1131
76. Sunderkotter C, Nikolic T, Dillon M J, Van Rooijen N, Stehling M, Drevets D A, Leenen P J: Subpopulations of mouse blood monocytes differ in maturation stage and inflammatory response, J Immunol 2004, 172:4410-4417
77. Torroella-Kouri M, Silvera R, Rodriguez D, Caso R, Shatry A, Opiela S, Ilkovitch D, Schwendener R A, lragavarapu-Charyulu V. Cardentey Y. Strbo N, Lopez DM: Identification of a subpopulation of macrophages in mammary tumor-bearing mice that are neither M1 nor M2 and are less differentiated, Cancer Res 2009, 69:4800-4809
78. Venet F, Huang X, Chung C S, Chen Y, Ayala A: Plasmacytoid dendritic cells control lung inflammation and monocyte recruitment in indirect acute lung injury in mice, Am J Pathol 176:764-773
79. Shechter R, London A, Varol C, Raposo C, Cusimano M, Yovel G, Rolls A, Mack M, Pluchino S, Martino G, Jung S. Schwartz M: Infiltrating blood-derived macrophages are vital cells playing an anti-inflammatory role in recovery from spinal cord injury in mice, PLoS Med 2009, 6:e1000113
80. Varol C, Landsman L, Fogg D K, Greenshtein L, Gildor B, Margalit R, Kalchenko V, Geissmann F, Jung S: Monocytes give rise to mucosal, but not splenic, conventional dendritic cells, J Exp Mcd 2007, 204:171-180

1. A method of treating a neurological disease and/or condition in a subject, comprising:
providing a therapeutically effective dosage of an immunological composition modified to express angiotensin converting enzyme (ACE), or a pharmaceutical equivalent, derivative, analog or salt thereof; and
administering the therapeutically effective dosage of the composition to the subject.

2. The method of claim 1, wherein the immunological composition comprises monocytes, macrophages and/or neuroglial cells.

3. The method of claim 1, wherein the immunological composition is administered to the subject intravenously.

4. The method of claim 1, wherein the immunological composition is administered to the subject by direct injection.

5. The method of claim 1, wherein the subject is a human.

6. The method of claim 1, wherein the subject is a rodent.

7. The method of claim 1, wherein the immunological compositions is administered to the subject by a bone marrow transfusion.

8. The method of claim 1, wherein the immunological composition has been derived from the bone marrow of the subject.

9. The method of claim 8, wherein the immunological composition has been modified in vitro to express ACE, or a pharmaceutical equivalent, derivative, analog or salt thereof.

10. The method of claim 1, wherein ACE or a pharmaceutical equivalent, derivative, analog or salt thereof, cleaves amyloid beta peptides in the subject.

11. The method of claim 1, wherein the neurological disease and/or condition is Alzheimer's disease.

12. The method of claim 1, wherein expression of ACE or a pharmaceutical equivalent, derivative, analog or salt thereof takes place in the brain of the subject.

13. A method of treating Alzheimer's disease in a subject, comprising:
providing a composition comprising a therapeutically effective dosage of angiotensin converting enzyme (ACE), or a pharmaceutical equivalent, derivative, analog or salt thereof; and
administering the composition to the subject.

14. The method of claim 13, wherein the composition is administered to the subject intravenously.

15. The method of claim 13, wherein the composition is administered to the subject by direct injection.

16. The method of claim 13, wherein the subject is a human.

17. The method of claim 13, wherein the subject is a rodent.

18. A composition comprising one or more cells genetically modified to selectively overexpress angiotensin converting enzyme (ACE) or a pharmaceutical equivalent, derivative, analog or salt thereof.

19. The composition of claim 18, wherein the one or more cells are myelomonocytic cells.

20. A pharmaceutical composition, comprising:
a therapeutically effective amount of angiotensin converting enzyme (ACE), or a pharmaceutical equivalent, derivative, analog or salt thereof; and
a pharmaceutically acceptable carrier.
